(19) United States
(12) Patent Application Publication
(10) Pub. No.: US 2005/0176151 A1
(43) Pub. Date: Aug. 11, 2005

(54) Freshness indicator of foodstuffs

(76) Inventor: Sang-Kyu Park, Gwangju (KR)

Correspondence Address: LADAS & PARRY LLP, 224 SOUTH MICHIGAN AVENUE, SUITE 1600, CHICAGO, IL, 60604, US

(21) Appl. No.: 10/516,814

(22) PCT Filed: Jun. 14, 2003
(86) PCT No.: PCT/KR03/01167

(30) Foreign Application Priority Data
Jun. 15, 2002 (KR) 10-2002-0333520

(51) Int. Cl. G01N 33/02

(52) U.S. Cl. 436/20

(57) ABSTRACT

The present invention relates to a freshness indicator of foodstuffs, and more particularly, to a freshness indicator of foodstuffs using pH sensitive high molecular substances which is capable of judging use (drinking) possibility, by indicating a quality of foodstuffs with naked eye using phase shift due to an ionization difference of pH sensitive high molecular substances which is sensitively reacted to pH of foodstuffs to be changed depending on a quality of foodstuffs, regardless of a term of validity marked on a vessel. The freshness indicator of foodstuffs comprises an ion and solvent through hole 10 in which an ion and solvent within foodstuffs to be changed depending on a quality of foodstuffs is formed such that it can be contacted with a pH sensitive high molecular substance, a semipermeable membrane member 20 being formed such that it passes an ion and solvent only through ion and solvent through hole 10, a filling layer 30 for a pH sensitive high molecular substance which is ionized or deionized by the ion and solvent being passed through semipermeable membrane member 20 and in which transparency/semitransparency phase shift is occurred, and a transparency film member 40 which is formed at the outside of filling layer 30 and is capable of judging a phase shift of filling layer with naked eye. Also, the freshness indicator of foodstuffs further comprises a freshness indication paper 50 in which a character or a figure is formed between filling layer 30 and semipermeable membrane member 20.

### TECHNICAL FIELD

The present invention pertains to a freshness indicator for foodstuffs, which indicates a quality of the foodstuffs using a phase shift of a pH sensitive high molecular weight substance caused by the ionization change of the pH sensitive high molecular weight substance sensitively depending on pH of the foodstuffs changed according to the quality of the foodstuffs, thereby allowing consumers to confirm the quality of the foodstuffs at a glance to judge consumability of the foodstuffs, regardless of a term of validity marked on a vessel containing the foodstuffs. More particularly, the present invention relates to a freshness indicator for foodstuffs including a semi-permeable membrane through which only ions and solvents of substances capable of passing through a hole penetrate, and a packed layer containing a pH sensitive high molecular weight substance ionized or deionized by the ions and solvents passing through the semi-permeable membrane to have a transparent phase or an opaque phase. At this time, the hole is formed so that the ions and solvents in the foodstuffs depending on a quality of the foodstuffs come into contact with the pH sensitive high molecular weight substance therethrough. A transparent film covers the packed layer so that consumers may easily observe a phase shift of the packed layer by looking therethrough. The freshness indicator further includes a freshness indicating paper having letters or figures drawn thereon and located between the packed layer containing the pH sensitive high molecular weight substance and the semi-permeable membrane.

### BACKGROUND ART

Generally, various foodstuffs such as livestock foodstuffs and dairy products deteriorate with time during circulation and storage, and differently change in quality according to a circulating or storing method. However, currently, a term of validity or preservation is indiscriminately marked on a vessel containing the foodstuffs, regardless of the circulating or storing method. Thus, the foodstuffs may presently only be judged by confirming the term of validity or preservation in terms of the freshness, and even the foodstuffs which rapidly deteriorate may be considered to be fresh so long as the term of validity has not expired.

In other words, consumers have no choice but to judge the freshness of the foodstuffs by confirming manufacturing date and the term of validity marked on the vessel containing the foodstuffs, and the term of validity is indiscriminately marked on the vessel containing the foodstuffs without taking into account a preservation state of the foodstuffs. Accordingly, the foodstuffs circulated or stored according to the wrong circulating or storing method may deteriorate and threaten consumers' health when the consumers eat the foodstuffs even though the term of validity has not expired. On the other hand, the foodstuffs are usually disposed of if the term of validity has expired even though they have been excellently circulated or stored so as to be fresh enough to be eaten by the consumers.

### DISCLOSURE OF THE INVENTION

Accordingly, the present invention has been made keeping in mind the above problems occurring in the prior art, and an aspect of the present invention is to provide a freshness indicator for foodstuffs using a pH sensitive substance, which indicates the freshness of the foodstuffs so as to allow consumers to judge consumability of the foodstuffs at a glance, regardless of the manufacturing date and preservation term of the foodstuffs.

Additional aspects and/or advantages of the invention win be set forth in part in the description which follows and, in part, will be obvious from the description, or may be learned by practice of the invention.

The above and/or other aspects are achieved by providing a freshness indicator for foodstuffs including a semi-permeable membrane through which only ions and solvents of substances capable of passing through a hole penetrate, and a packed layer containing a pH sensitive high molecular weight substance ionized or deionized by the ions and solvents passing through the semi-permeable membrane to have a transparent phase or an opaque phase. At this time, the hole is formed so that the ions and solvents in the foodstuffs, depending on a quality of the foodstuffs, come into contact with the pH sensitive high molecular weight substance therethrough. A transparent film covers the packed layer so that consumers may easily observe a phase shift of the packed layer by looking therethrough. The freshness indicator further includes a freshness indicating paper having letters or figures drawn thereon and located between the packed layer containing the pH sensitive high molecular weight substance and the semi-permeable membrane. Therefore, the freshness indicator of the present invention contributes to allowing consumers to easily confirm a quality of the foodstuffs at a glance to judge consumability of the foodstuffs, regardless of a term of validity marked on a vessel containing the foodstuffs.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a sectional view of a freshness indicator according to the present invention;

FIG. 2 schematically illustrates an operation mechanism of the freshness indicator according to an embodiment of the present invention;

FIG. 3 is a graph showing transparency of a pH sensitive material as a function of pH of foodstuffs for the freshness indicator according to the present invention; and

FIG. 4 illustrates the freshness indicator of the present invention applied to the products.

### BEST MODE FOR CARRYING OUT THE INVENTION

Reference should now be made to the drawings, in which the same reference numerals are used throughout the different drawings to designate the same or similar components.

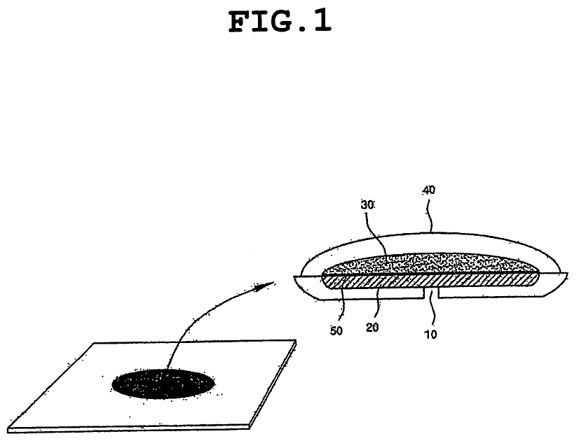

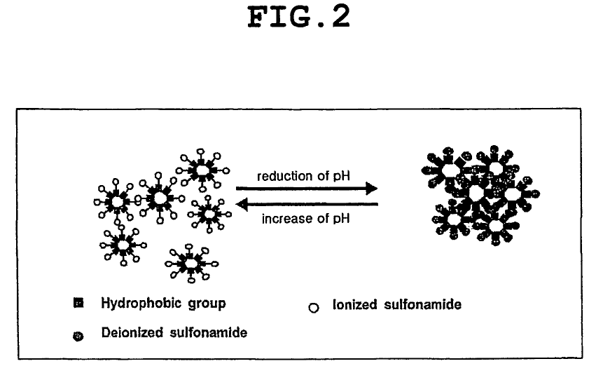

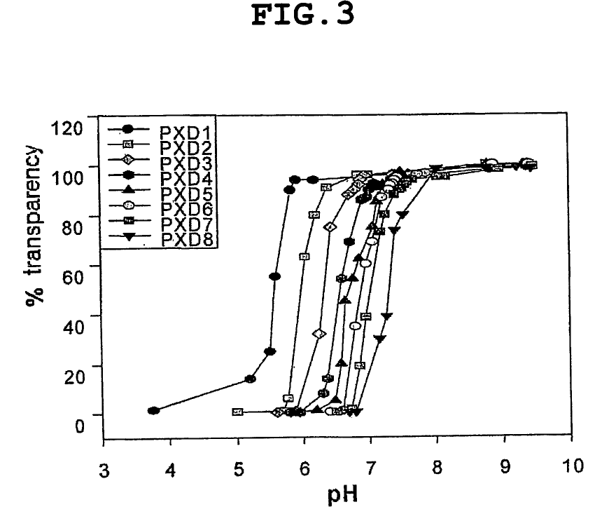

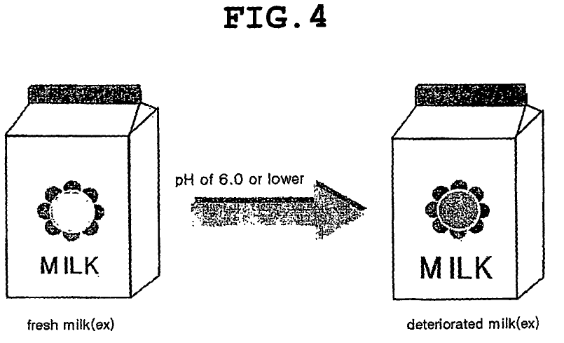

As shown in the drawings, FIG. 1 illustrates a sectional view of a freshness indicator according to an embodiment of the present invention, FIG. 2 schematically illustrates an operation mechanism of the freshness indicator according to the present invention, FIG. 3 is a graph showing transparency of a pH sensitive material as a function of pH of foodstuffs for the freshness indicator according to the present invention, and FIG. 4 illustrates the freshness indicator applied to the products.

The freshness indicator for foodstuffs according to the present invention includes a semi-permeable membrane 20 through which only ions and solvents of substances capable of passing through a hole 10 penetrate, and a packed layer 30 containing a pH sensitive high molecular weight substance ionized or deionized by the ions and solvents passing through the semi-permeable membrane 20 to have a transparent phase or an opaque phase. At this time, the hole 10 is formed so that the ions and solvents in the foodstuffs depending on a quality of the foodstuffs come into contact with the pH sensitive high molecular weight substance therethrough. A transparent film 40 covers the packed layer 30 so that consumers may easily observe a phase shift of the packed layer 30 by looking therethrough. The freshness indicator further includes a freshness indicating paper 50 having letters or figures drawn thereon and located between the packed layer 30 containing the pH sensitive high molecular weight substance and the semi-permeable membrane 20.

With reference to FIG. 1, the freshness indicator is attached to a packing paper of the foodstuffs, and includes a semi-permeable membrane 20 through which only ions and solvents of substances capable of passing through a hole 10 may penetrate, and a packed layer 30 containing a substance sensitive to a specific pH ionized or deionized by the ions and solvents passing through the semi-permeable membrane 20 to have a transparent phase or an opaque phase.

At this time, the hole 10 is formed so that the ions and solvents in the foodstuffs depending on a quality of the foodstuffs come into contact with the pH sensitive high molecular weight substance therethrough.

The transparent film 40 is positioned covering the packed layer 30, and consumers confirm the phase shift of the packed layer 30 through the transparent film 40 by looking. Additionally, the freshness indicating paper 50 on which letters or figures are drawn is positioned between the packed layer 30 containing the pH sensitive high molecular weight substance and the semi-permeable membrane 20.

Any semi-permeable membrane may be used so long as it allows only the ions and solvents contained in the foodstuffs to penetrate therethrough. Further, sulfonamide reacts with methacryloyl chloride to produce an ionized sulfonamide group, and the ionized sulfonamide group thus produced reacts with N,N-dimethylacrylamide which is included in acrylamides in various molar ratios to produce plural pH sensitive high molecular weight substance samples.

Examples of usable sulfonamides include sulfadiazine, sulfabenzamide, sulfacetamide, sulfisoxazole, sulfamethizole, sulfadimethoxine, sulfapyridine, sulfamethazine, sulfisomidine, and sulfamethoxypyridazine.

A procedure of producing a pH sensitive monomer using sulfadiazine is shown in the following Reaction equation 1.

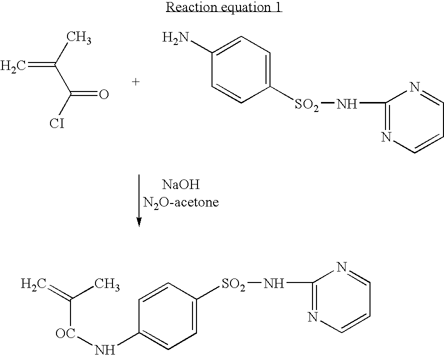

Furthermore, a procedure of reacting the pH sensitive monomer, for instance a sulfadimethoxine monomer, with N,N-dimethylacrylamide which is an acrylamide-based hydrophobic monomer to produce the pH sensitive high molecular weight substance is shown in the following Reaction equation 2.

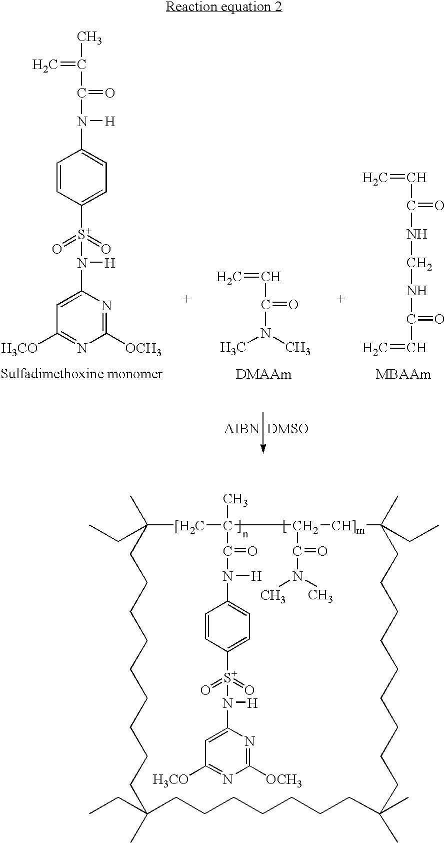

In the Reaction equation 2, MBAAm is a crosslinker, and AIBN is an initiator.

Meanwhile, after the pH sensitive monomer reacts with N,N-dimethylacrylamide which is the acrylamide-based hydrophobic monomer in various molar ratios to produce plural pH sensitive high molecular weight substance samples, the pH sensitive high molecular weight substance most suitable in a desired pH range is selected.

For example, sulfadiazine (methacryloyl chloride) which is the pH sensitive monomer may be reacted with N,N-dimethylacrylamide in molar ratios of 1:9, 2:8, and 3:7. At this time, a swelling ratio of the pH sensitive high molecular weight substance depends on the molar ratio, thus the molar ratio change affects the transparency of the pH sensitive high molecular weight substance.

The swelling ratio of the pH sensitive high molecular weight substance denotes the degree of water held in the pH sensitive high molecular weight substance. Accordingly, if the swelling ratio of the pH sensitive high molecular weight substance is low at pH 5 and is high at pH 6, the pH sensitive high molecular weight substance holds more water at pH 6 than at pH 5. At this time, the swelling ratio change of the pH sensitive high molecular weight substance causes the transparency change of the pH sensitive high molecular weight substance.

Additionally, a variable pH range of the pH sensitive high molecular weight substance is proportionally increased with a sulfadiazine (methacryloyl chloride) content in the pH sensitive high molecular weight substance. From the following Table 1, it can be seen that when sulfadiazine reacts with N,N-dimethylacrylamide in a molar ratio of 1:9, the variable pH range is about six, but when sulfadiazine reacts with N,N-dimethylacrylamide in a molar ratio of 2:8, the variable pH range is six or higher. Therefore, the variable pH range is controlled using the molar ratio of sulfadiazine(methacryloyl chloride) which is the pH sensitive monomer and N,N-dimethylacrylamide.

TABLE 1
Swelling ratio

| Molar ratio/pH | 5 | 6 | 7 | 8 |
|---|---|---|---|---|
| 1:9 | 10.4 | 11.22 | 20.47 | 28.67 |
| 2:8 | 10.4 | 14 | 24 | 68 |
| 3:7 | 3.8 | 4.3 | 9.8 | 43 |

In FIG. 3, PXD 1 denotes that sulfadiazine(methacryloyl chloride) which is the pH sensitive monomer reacts with N,N-dimethylacrylamide in a molar ratio of 1:9, and PXD 2 to PXD 8 respectively denote that sulfadiazine(methacryloyl chloride) reacts with N,N-dimethylacrylamide in molar ratios of 2:8, 3:7, 4:6, 5:5, 6:4, 7:3, and 8:2.

A detailed description will be given of the operation of the freshness indicator for the foodstuffs using the pH sensitive high molecular weight substance according to the present invention. When the preservation term of livestock foodstuffs or dairy products expires, the livestock foodstuffs or dairy products are deteriorated, thereby being changed in their pH (acidity). In this regard, ions or solvents contained in the livestock foodstuffs or dairy products which are changed in terms of pH penetrate through the semi-permeable membrane 20 into the packed layer 30 including the pH sensitive high molecular weight substance, thereby the pH change of the livestock foodstuffs or dairy products brings about the phase shift of the packed layer 30 including the pH sensitive high molecular weight substance.

For example, usually, when the pH of milk is 5.8 or lower, it is not suitable to drink, and the pH of the milk is lowered when the milk is deteriorated due to the wrong circulating or storing method regardless of the term of validity. At this time, the ions and solvents contained in the deteriorated milk advance through a semi-permeable membrane into the packed layer containing the pH sensitive high molecular weight substance, thus the pH sensitive high molecular weight substance is deionized and becomes opaque as shown in FIGS. 2 and 3. Hence, consumers may easily confirm whether the milk is fresh or not as shown in FIG. 4.

Furthermore, in the present invention, the freshness indicating paper 50 on which the letters or figures are drawn is located between the packed layer 30 containing the pH sensitive high molecular weight substance and the semi-permeable membrane 20 so as to easily discriminate the freshness of the foodstuffs. Accordingly, when the foodstuffs are fresh, the pH sensitive high molecular weight substance is transparent, thus the letters or figures of the freshness indicating paper 50 may be easily observed by the consumers and the consumers can confirm the freshness of the foodstuffs by simply looking.

According to the present invention, the freshness indicating paper 50 is applied only to a packing paper. However, an application field of the freshness indicating paper 50 is not limited by the packing paper, and it may be produced in a form of an independent kit.

### INDUSTRIAL APPLICABILITY

As apparent from the above description, the present invention provides a freshness indicator for foodstuffs including a semi-permeable membrane through which only ions and solvents of substances capable of passing through a hole penetrate, and a packed layer containing a pH sensitive high molecular weight substance ionized or deionized by the ions and solvents passing through the semi-permeable membrane to have a transparent phase or an opaque phase. At this time, the hole is formed so that the ions and solvents in the foodstuffs depending on a quality of the foodstuffs come into contact with the pH sensitive high molecular weight substance therethrough. A transparent film covers the packed layer so that consumers may easily observe a phase shift of the packed layer by looking therethrough. The freshness indicator further includes a freshness indicating paper having letters or figures drawn thereon and located between the packed layer containing the pH sensitive high molecular weight substance and the semi-permeable membrane. Therefore, the freshness indicator attached to a packing paper used to pack the foodstuffs or provided in a form of an independent kit contributes to allowing consumers to easily confirm a quality of the foodstuffs by their naked eyes to judge consumability of the foodstuffs, regardless of a term of validity marked on a vessel containing the foodstuffs.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A freshness indicator for foodstuffs using a pH sensitive high molecular weight substance, comprising:
a semi-permeable membrane through which only ions and solvents of substances capable of passing through a hole penetrate, the hole being formed so that the ions and solvents in the foodstuffs depending on a quality of the foodstuffs come into contact with a pH sensitive high molecular weight substance therethrough;
a packed layer containing the pH sensitive high molecular weight substance ionized or deionized by the ions and solvents passing through the semi-permeable membrane to have a transparent phase or an opaque phase; and
a transparent film covering the packed layer so that consumers easily observe a phase shift of the packed layer by looking therethrough.

2. The freshness indicator as set forth in claim 1, further comprising a freshness indicating paper having letters or figures drawn thereon and located between the packed layer containing the pH sensitive high molecular weight substance and the semi-permeable membrane.

3. The freshness indicator as set forth in claim 1, wherein the pH sensitive high molecular weight substance is produced by reacting an ionized sulfonamide group with N,N-dimethylacrylamide as an acrylamide-based hydrophobic monomer after sulfonamide reacts with methacryloyl chloride to produce the ionized sulfonamide group.

4. The freshness indicator as set forth in claim 3, wherein the sulfonamide is selected from the group consisting of sulfadiazine, sulfabenzamide, sulfacetamide, sulfisoxazole, sulfamethizole, sulfadimethoxine, sulfapyridine, sulfamethazine, sulfisomidine, and sulfamethoxypyridazine.
